(19) United States
(12) Patent Application Publication
(10) Pub. No.: US 2004/0136870 A1
(43) Pub. Date: Jul. 15, 2004

(54) Automatic analysis apparatus

(76) Inventors: Thomas E. Kochy, San Jose, CA (US); Joseph I. Keto, San Francisco, CA (US); Vidal O. Smith, Tracy, CA (US); Roger A. O'Neill, San Carlos, CA (US)

Correspondence Address: Aldo J. Test, DORSEY & WHITNEY LLP, Suite 3400, 4 Embarcadero Center, San Francisco, CA, 94111, US

(21) Appl. No.: 10/693,080

(22) Filed: Oct. 24, 2003

Related U.S. Application Data
(60) Provisional application No. 60/421,448, filed on Oct. 25, 2002.

(51) Int. Cl. G01N 33/00

(52) U.S. Cl. 422/73

(57) ABSTRACT

An automatic analyzing apparatus in which liquid samples contained in an array of cuvettes or wells are presented sequentially to a suspended capillary of a particle analyzing apparatus. The analyzing apparatus includes a mechanism which has a support tray for supporting the array of cuvettes or wells and moves the tray in the x-y direction to bring individual cuvettes opposite the capillary and then lifts the tray to immerse the capillary in the liquid sample of the selected cuvette or well for sample analysis.

### RELATED APPLICATIONS

This application claims priority to provisional application Serial No. 60/421,448 filed Oct. 25, 2002.

### BRIEF DESCRIPTION OF THE INVENTION

The present invention is directed to an apparatus for performing analysis of cells or particles in a liquid sample and more particularly an apparatus which sequentially presents liquid samples contained in an array of microwells or cuvettes to the analyzing apparatus.

### BACKGROUND OF THE INVENTION

The detection and analysis of individual particles or cells is important in medical and biological research. It is particularly important to be able to measure characteristics of particles such as concentration, number, viability, identification and size. Individual particles or cells are herein defined to include, for example, bacteria, viruses, DNA fragments, cells, molecules and constituents of whole blood.

One type of apparatus for analyzing particles or cells is a flow cytometer. In flow cytometry, particles that are either intrinsically fluorescent or labeled with a fluorescent marker are caused to flow past a beam of radiant energy. The radiant energy excites the particles or marker which emit a characteristic color (wavelength) of fluorescent light. One or more photodetectors detect the fluorescent light emitted by the particles or labels at selected wavelengths as they flow through the light beam and generate output signals representative of the particles. In most cytometers a photodetector is also used to measure forward scatter of the light to generate signals indicative of the presence and size of particles.

In co-pending application Ser. No. 09/844,080 filed Apr. 26, 2001, incorporated in its entirety herein by reference, there is described a particle or cell analyzer and method and more particularly a particle or cell analyzer and method in which the sample solution containing the particles or cells is drawn from a well or cuvette through a suspended capillary for presentation to the excitation light beam.

The present invention is directed to an apparatus for presenting fluid sample contained in an array of cuvettes or wells carried by a microwell plate or cuvette rack one at a time to the end of the capillary, where the sample is aspirated and flows through the capillary past the excitation light beam. In order to better understand operation of the present invention, the cell analyzer described in said co-pending application is shown and briefly described with reference to FIGS. 1 and 2. Referring to FIG. 1, fluid sample 11 is drawn or aspirated from the well or cuvette 12 into capillary 13. In order to identify and count particles as they traverse the light beam 14 the volume of fluid must be correlated with the number of particles detected. The sample is drawn through the capillary tube at a constant rate by an electrically operated calibrated syringe or pump 16. The pump may be any other type of pump such as the parastoltic pump which can draw known volumes of samples through the capillary past the light beam. The pump is connected to the capillary tube by a conduit 17. This permits changing capillaries 13 to substitute a clean capillary or a capillary having a different diameter, as needed for various types and sizes of particles or cells. As illustrated, the pump comprises a syringe pump 16, which draws sample fluid into the capillary by moving the plunger 19. The pump 16 is also connected to waste or drain conduit 21, which includes a valve 22. When the valve is closed, the pump draws sample from the well or cuvette through the capillary tube 13 past the light beam 14. After an analysis has been completed, the valve 22 is opened, whereby reversal of direction of plunger 19 causes fluid to flow through the waste conduit 21 into the waste container 23. The diameter of the waste tube or conduit 21 is selected to be many times, 10 or more than that of the capillary, whereby substantially all of the fluid from the syringe is discharged into the waste. For example, if there is a factor of ten ratio in diameter, only 1/10,000 of the fluid will travel back through the capillary, a negligible amount.

The syringe pump is designed so that a predetermined movement of the plunger 19 will draw a known volume through the capillary and past the light beam. The pump can be calibrated for each size capillary by drawing a fluid into the pump and by moving the plunger a known distance and then discharging and measuring the discharged fluid volume. Thereafter, for a given movement of the plunger the volume of the sample which flows past the light beam is known. The volume can either be determined by measuring movement of the plunger or measuring the time the plunger is moved if it is calibrated as a function of time.

FIG. 2 schematically illustrates the optical system for exciting a volume of fluid in the capillary and provide output scattered light representing the number and fluorescent light characteristics of the types of particles passing through the analyzer volume. Light beam 14 preferably a laser beam emits light at selected wavelength. The light is received by an optical focusing system (not shown) which focuses said light and forms and directs a beam through the analyzing volume. In order to count all particles which pass through the analyzing volume, that is, particles which are tagged to fluoresce and untagged particles, scattered light is detected. In one embodiment, a beam blocker 27 is positioned to intercept the direct beam after it passes through the capillary tube. This allows only light scattered by the particle to impinge onto detector 28. The detector 28 provides an output signal pulse each time a particle passes through the beam. The size of the pulse is dependent upon the size of the particles and the occurrence of a pulse indicates that a particle (fluorescent or non-fluorescent) has traversed the light beam. Another approach is to employ an off axis detector 29. In such event, a beam blocker is not required.

If the particles are intrinsically fluorescent or if the particles have been tagged with a fluorescent dye, they will emit light at a characteristic wavelength as they pass through the analyzing volume where they are excited and fluoresce. Fluorescent light is detected at an angle with respect to the beam axis so that no direct beam light is detected. Collector lens 32, 33 and 36, 37 receive the emitted light and focus the collected light onto detectors 38, 39 and 41, respectively. Spatial filters 42, 43 and 44 may be provided to block any stray light. Dichroic beam splitter 46 passes light of the selected wavelength through filter 47 to detector 38 and deflects light of other wavelengths through filter 48 to photodetector 49. For example, the dichroic beam splitter reflects light having a wavelength less than 620 nm, and transmits light having a greater wavelength. The filters 47 and 49 are selected to pass the wavelengths corresponding to the fluorescence wavelength expected from the fluorescing particles. In one example, the filters 47 and 49 were selected to pass light at 580 nm and 675 nm, respectively. Fluorescent light can be applied to filter 51 and detector 41 for detecting another wavelength. This permits identification and counting of particles which have been tagged with fluorescent material which emits at characteristic wavelengths in response to the optical excitation. The output of the detector are pulses which can then be processed by a processor to provide relevant information.

It is desirable to process a large number of samples in a given time period with a single instrument. Therefore, there is a need for a cytometer that automatically and sequentially draws or aspirates sample liquid from sample wells in a sample carrier. One type of sample carrier is a microwell plate. A microwell plate is typically a plastic plate containing an array of uniformly spaced cavities or wells for holding the sample fluid. One type of commercial microwell plate is a 96 well plate which contains an array of eight rows of 12 wells. Other sizes are commercially available. Samples can also be contained in cuvettes which are held in rows and columns by suitable racks. In some cases it is desirable to mix or stir the sample liquid just prior to drawing or aspirating the liquid sample.

### OBJECTS AND SUMMARY OF THE INVENTION

The primary object of the present invention is to provide an automatic analyzing apparatus for fluorescent analysis of cells or particles in liquid samples contained in an array of cuvettes or wells which are presented automatically and sequentially to a capillary tube for aspiration of liquid sample into and through the tube past an analyzing region.

It is another object of the present invention to provide an analysis apparatus in which a mechanism moves individual cuvettes or wells in an array to a position opposite a suspended capillary and raises the cuvette or well so that the tip of the capillary is immersed in the liquid sample for aspiration of the liquid samples into and through the capillary.

It is another object of the present invention to provide a mechanism capable of presenting fluid in individual cuvettes or wells to a suspended capillary accurately and at high speed.

It is the further object of the present invention to provide an automatic analyzing apparatus which is simple in construction.

It is the further object of the present invention to provide such an apparatus which includes means for mixing or stirring fluid prior to its being sampled or aspirated.

Still another object of the present invention is to provide a cytometer apparatus which can perform analysis more quickly than standard cytometers.

The apparatus uses a suspended capillary through which sample is aspirated past an analyzing zone. This allows the sample particles or cells to pass an analyzing zone one at a time where they can be individually analyzed. The invention provides a translation mechanism which moves microwells or cuvettes carried in a rack selectively into position below a suspended capillary and raises the positioned well or cuvette so that the end of the capillary is immersed in the sample liquid such that the sample can then be aspirated or drawn into the capillary past the analyzing region. This eliminates the handling of individual cuvettes or wells which requires skilled operators and is time consuming.

In another aspect of the invention the apparatus includes a mixer which mixes or stirs the sample fluid in the wells just prior to presentation to the capillary.

An analyzing apparatus is provided which includes a mechanism for sequentially translating individual sample containers into position opposite the end of a suspended capillary and then raising the sample container to immerse the end of the capillary so that the liquid can be drawn or aspirated into the capillary for analysis.

### BRIEF DESCRIPTION OF THE DRAWINGS

The invention will now be described in greater detail with reference to the accompanying drawings in which:

FIG. 1 schematically shows the drawing of a fluid sample from a cuvette or well into and through a capillary past an analyzing region;

FIG. 2 is a schematic diagram illustrating the optical system associated with the capillary for exciting fluorescence in particles passing through an analyzing region and for detecting fluorescent light at various wavelengths;

FIG. 3 is a partial side elevational view partly in section showing the sampling portion of an automatic analysis apparatus;

FIG. 4 is a perspective view showing the mechanism for moving a support tray with cuvettes or wells carried by a plate into position and into cooperation with a suspended capillary;

FIG. 5 is an elevational view showing the cam mechanism for lifting the support tray to immerse the end of the capillary into the sample fluid carried in wells or cuvettes;

FIG. 6 is a sectional view showing the cam follower for raising and lowering the support tray;

FIGS. 7A, 7B, and 7C show three positions for the tray with respect to a stirrer or mixer and suspended capillary;

FIG. 8 shows a stirrer assembly which surrounds the capillary;

FIG. 9 shows electromagnetic means for vibrating the capillary to mix samples;

FIG. 10 is a partial elevational view showing analyzing apparatus employing two capillaries; and

FIG. 11 is a schematic view of an electrical circuit for operating the apparatus.

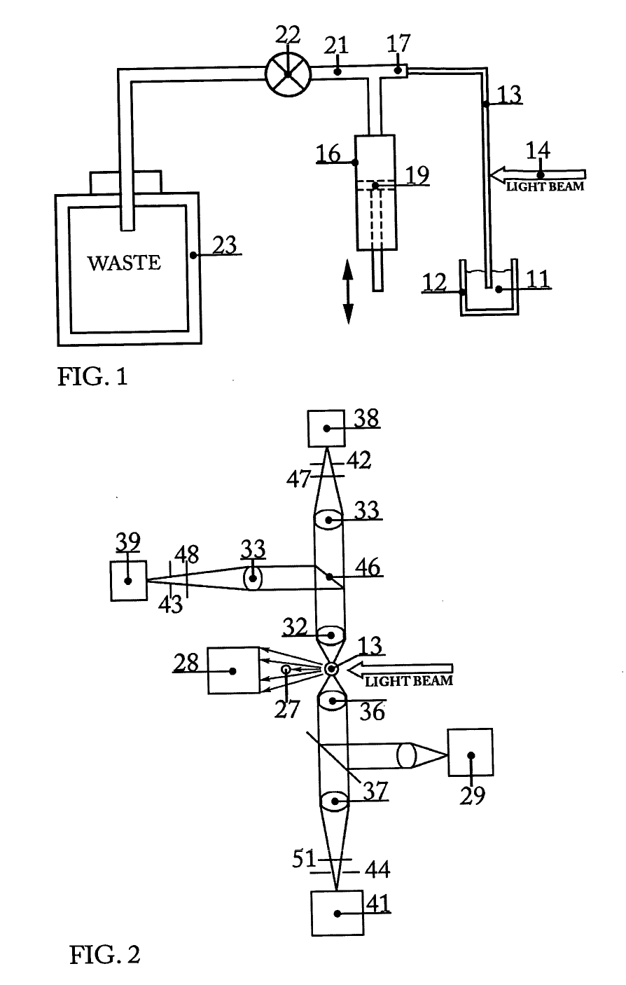

### DESCRIPTION OF THE PREFERRED EMBODIMENT

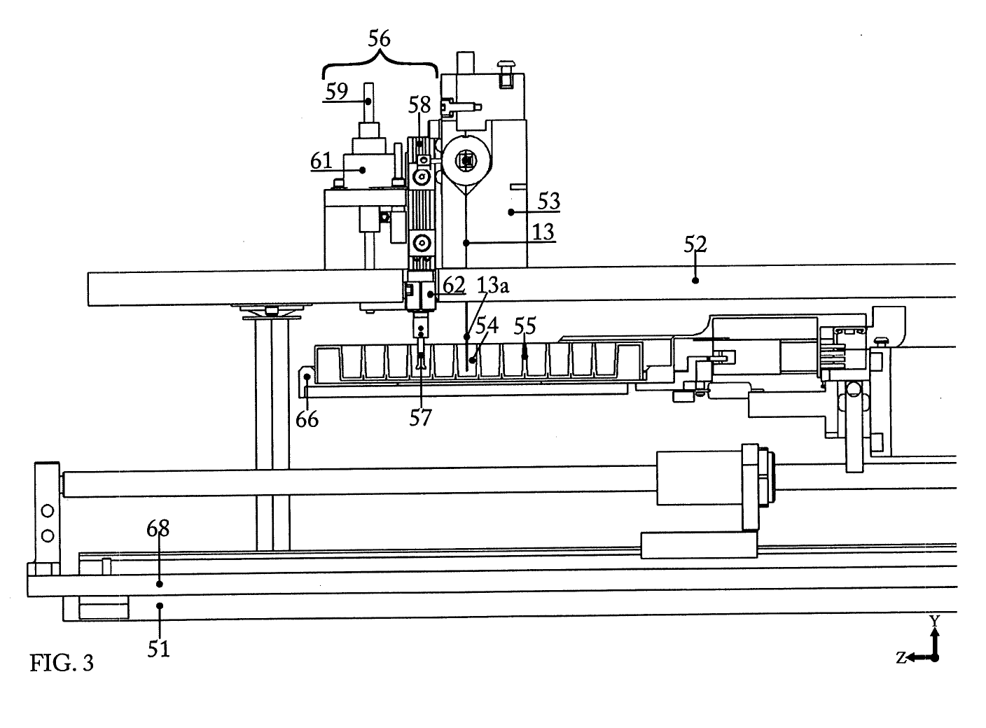

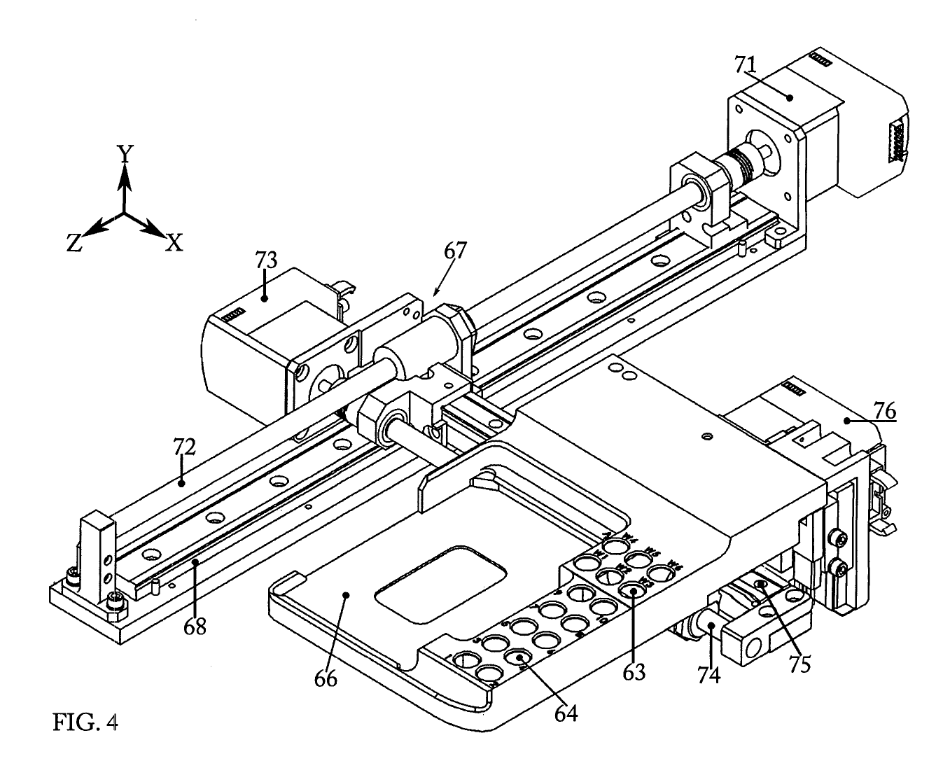

Referring now particularly to FIGS. 3 and 4, the apparatus includes a base 51 on which the positioning mechanism, FIG. 4, is mounted. A shelf 52 is supported from and above the base. The shelf 52 carries the optical elements (not shown) but described with reference to FIG. 2. The suspended capillary 13 extends downwardly from the shelf and is schematically shown supported by a support 53. A suitable support assembly which provides for removal and insertion of capillaries of various sizes is described in co-pending application Ser. No. 10/146,019 filed May 14, 2002, incorporated herein by reference. The capillary is suspended with its end 13a shown extending into microwell 54 in microwell plate 55.

The apparatus may include mixer or stirrer assembly 56 supported on the shelf. The stirrer assembly includes a downwardly extending stirrer 57 which is raised and lowered along the rail 58 by a lead screw 59 driven by a motor 61. A mixer motor 62 is selectably energized to rotate the stirrer 57 to stir or mix the liquid samples in the associated microwell or cuvette. The stirrer is shown stirring liquid samples in well, two wells in front of the well 54 which is being sampled. The microwell plate 55 is supported on the support tray 66 which is part of the mechanism, FIG. 4, for moving the tray in the X, Y and Z directions.

Referring to FIG. 4, the support tray 66 includes wash wells 63 which may be filled with a wash solution. When it is desired to clean the capillary after one or more sampling cycles, the tray is moved to a position such that the wash well is opposite the capillary and raised to immerse the capillary for cleaning. The tray also includes waste collection tubes 64 which are adapted to receive the stirrer. The stirrer is cleaned by inserting it into one of the collection tubes and rapidly spinning it.

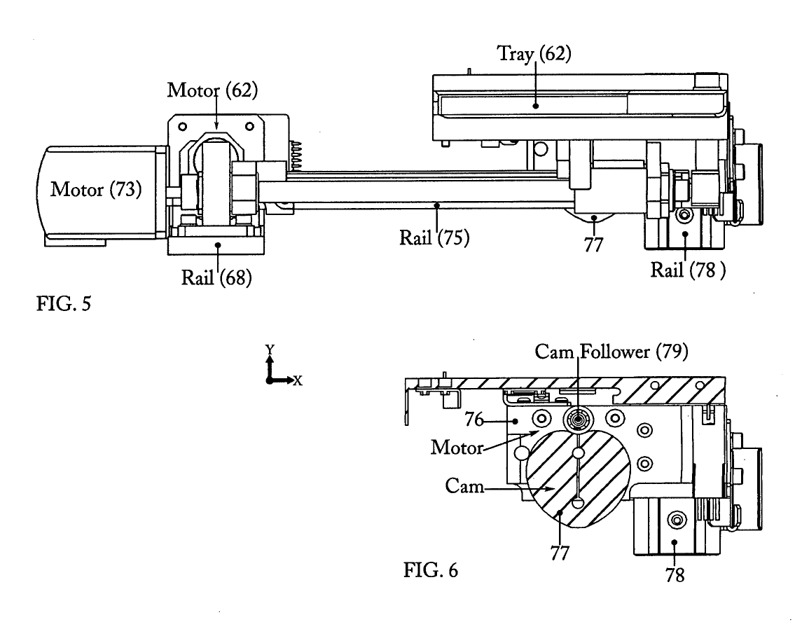

Referring more particularly to FIG. 4 the tray is carried by an assembly 67 which is supported upon a rail 68 mounted on the base 51. The assembly is driven in the X direction by the motor 71 driving lead screw 72. The support assembly includes a second motor 73 and associated lead screw 74 which drives the tray 62 in the Y direction along the rail 75. The support assembly also includes a cam means that moves along the rail 75 for lifting the support tray. The cam assembly is more clearly shown in FIGS. 5 and 6 and includes a motor 76 which drives the cam 77 which is rotated to lift the tray and move it along the rail 78 via the cam follower 79 which provides the lifting mechanism. Thus, there is provided a mechanism for selectively moving the tray in the X, Y and Z directions.

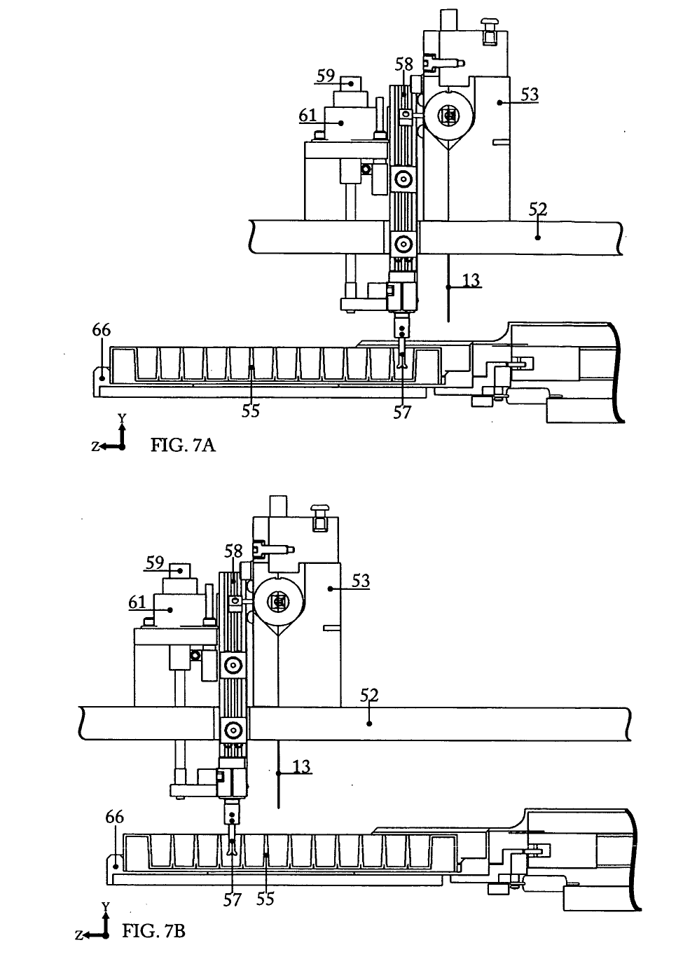

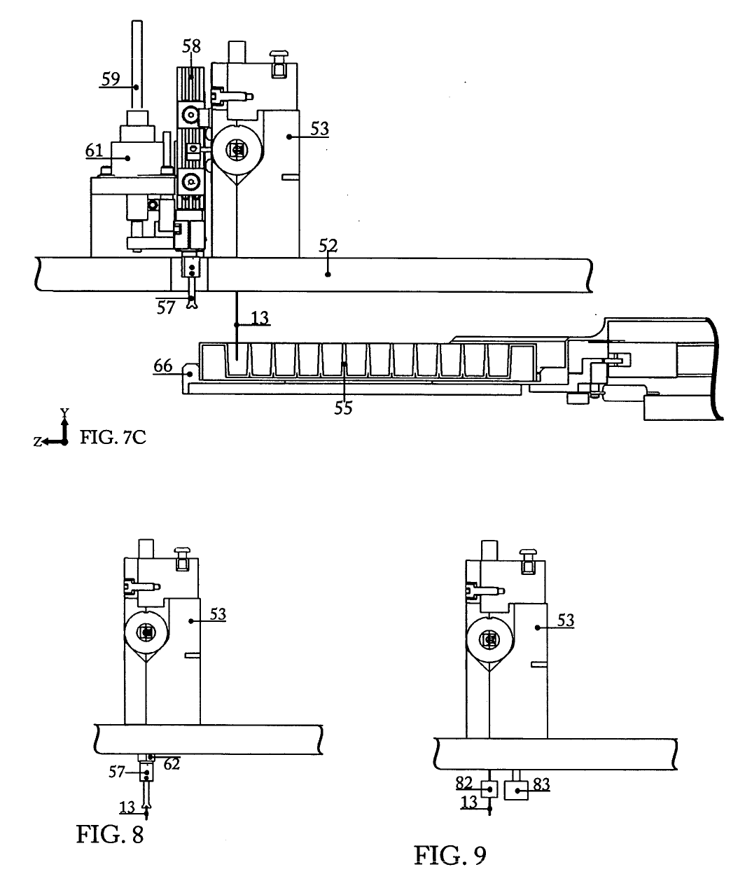

In operation, the support is moved in the X direction so that it extends outwardly from the instrument housing (not shown) where a technician or operator can place a microwell plate or a cuvette rack onto the tray 66. The tray is then retracted to the position shown in FIG. 7A and the analyzing process commenced. To stir the sample liquid in the first few wells the tray is in its lowered position and the stirrer 57, in its lowest position in first well, to stir the sample liquid. It is then raised and the tray moved over one position. The stirrer is then moved into the second well to stir liquid in its second well. Since the tray is in its lowered position it can move without striking the suspended capillary 13. Thereafter, the mixer is retracted to a position with its end at the same elevation as the end of the capillary, FIG. 7B. The tray is then moved one step at the time and raised so that the capillary is immersed in the liquid in the first well while the mixer mixes the liquid in the third well. The tray is then lowered, moved one step, raised to sample the liquid in the next well and mix the fluid in the next succeeding well. The step-wise procedure is continued until the end of the tray at which time during the last two sampling steps the mixer is lifted as shown in FIG. 7C whereby it does not interfere with the tray, as it moves while the capillary can still be immersed in the liquid to withdraw liquid. After sampling one row of wells, the tray is lowered, moved in the Y direction by one row and the procedure is repeated. It is of course apparent that the wells can be sampled column by column by stepping from well to well in the Y direction and then stepping to the next column in the X direction. It also will be apparent the apparatus can be programmed to sample selected wells in selected sequences. The mixer and/or capillary can be washed periodically by moving the tray in the X-Y direction to a wash or cleaning well 63 and moving the tray in the Z direction to immerse the capillary. Similarly, the mixer can be cleaned by X, Y and Z movement of the tray so that the mixer is within a tube 64.

The sample can be mixed by vibrating the tray rather than with a stirrer. Alternatively, the stirrer can surround the capillary as shown in FIG. 8 and rotated by motor 62 whereby it can mix the sample in the selected well. Mixing can also be accomplished by vibrating the capillary as for example in FIG. 9 by applying a magnetic member 82 to the capillary and magnetically deflecting it with an energizing coil 83.

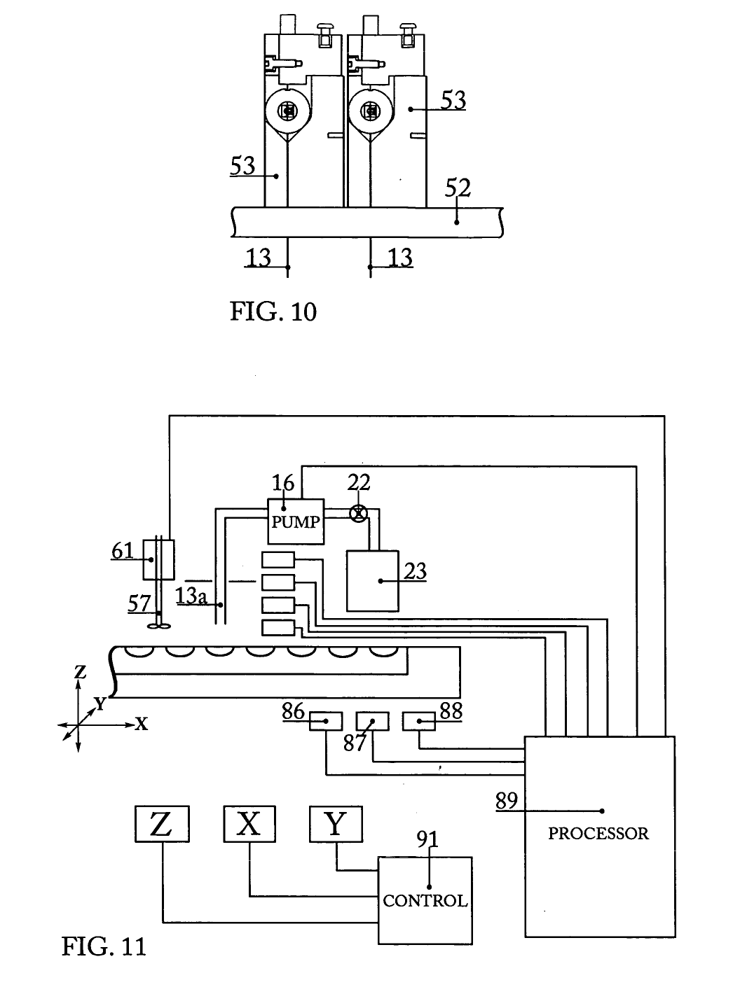

Rather than using a single capillary, an optical system using multiple capillaries and multiple optical systems can be mounted on the shelf 52 to conduct sample analysis in parallel. This is illustrated schematically in FIG. 10 where two side-by-side capillaries are illustrated.

FIG. 11 schematically illustrates the electrical control circuit for the preferred embodiment automatic analysis apparatus. X, Y and Z position sensors 86, 87 and 88 mounted on the positioning mechanism to sense the position of the tray and feed the position information to the processor 89. The processor controls the pump 16 and valve 22 to sequentially withdraw sample fluid from a well or cuvette associated with the end of the capillary and discharge fluid samples into waste 23. The processor controls the stirrer motor for energizing the stirrer 57 to rotate the stirrer and the stirrer motor 61 for raising and lowering the stirrer and the stirrer motor 62. The outputs of the photodetectors 28, 38, 39 and 41 are also applied to the processor. The processor commands motor control 91 associated with the X and Y drive motors and the Z cam motor whereby they are selectively energized to position the tray. In operation, the tray is loaded with the microwell plate or cuvette rock and the drive mechanism energized to bring the tray into its initial position. The analysis cycle is then started in which stirrer is lowered and stirs or mixes the fluid sample in the first two vials or wells. The stirrer is then withdrawn to be in the same position as the end of the capillary. The tray is then lifted whereby the capillary is immersed in the sample of the first well and the stirrer in the third well to perform mixing at this point. The pump is turned on and a sample is drawn through the capillary and analyzed by detecting the fluorescence and the forward scatter for a predetermined volume of sample liquid. The sample liquid is then discharged into the waste. The tray is then lowered and moved one position to the right and the process is repeated as disclosed above. The processor performs automatic fluorescent analysis of the sample liquid. It will be recognized that the processor can be configured for wash and cleaning cycles and for controlling the tray for multiple capillaries.

Thus there has been provided a simple inexpensive mechanism for presenting sample liquid to cytometer apparatus of the type having a syspended capillary. The mechanism automatically and sequentially presents sample wells or cuvettes to a suspended capillary where solution is aspirated and analyzed.

What is claimed is:

1. A mechanism for presenting individual liquid samples carried by spaced wells in a microwell plate or cuvettes in a rack to the end of a suspended capillary comprising:
a support tray for said supporting plate or rack,
x-axis drive means and y-axis drive means functionally associated with said support tray for moving said tray in the x and y directions to bring a selected well or cuvette opposite the suspended capillary,
z-axis drive means functionally associated with said tray for moving said support tray in the z direction to immerse the end of the capillary in a liquid sample carried in said well or cuvette; and
drive control means for controlling the z-axis drive means to lower the support tray when the support tray is moved in the x or y directions so that it can move without damaging the suspended capillary and raising the support tray to bring the liquid in a selected cuvette or microwell into cooperation with the end of the capillary.

2. A mechanism as in claim 1 in which the z-axis drive means comprises a motor driven cam.

3. A mechanism as in claims 1 or 2 in which the x-axis and y-axis drive means comprise motor driven lead screws.

4. A cytometer system in which sample liquid is drawn from a sample container in an array of sample containers past an analyzing volume in a capillary by immersing the suspended end of the capillary in the sample liquid and drawing the liquid sample past the analyzing volume including
a mechanism for sequentially translating individual sample containers to a position opposite the end of the suspended capillary and then lifting the container to immerse the end of the capillary in the liquid so that the liquid can be drawn through the capillary.

5. A cytometer system as in claim 4 including a stirrer adapted to stir the liquid in a sample container.

6. A mechanism for mixing and presenting individual liquid samples carried by spaced wells in a microwell plate or cuvettes in a rack to the end of a suspended capillary comprising:
a mixing means associated with said capillary;
a support tray for said supporting plate or rack,
x-axis drive means and y-axis drive means functionally associated with said support tray for moving said tray in the x and y directions to bring a selected well or cuvette opposite the suspended capillary,
z-axis drive means functionally associated with said tray for moving said support tray in the z direction to immerse the end of the capillary in a liquid sample carried in said well or cuvette; and
drive control means for controlling the z-axis drive means to lower the support tray when the support tray is moved in the x or y directions so that it can move without damaging the suspended capillary, and mixing means and raising the support tray to bring the liquid in a selected cuvette or microwell into cooperation with the mixing means and the end of the capillary.

7. A mechanism as in claim 6 in which the z-axis drive means comprises a motor driven cam.

8. A mechanism as in claims 6 or 7 in which the x-axis and y-axis drive means comprise motor driven lead screws.

9. A mechanism as in claim 6 in which the mixing means is adjacent the capillary and includes means for moving the mixes in the z direction.

10. A mechanism as in claim 6 in which the mixing means is coaxial with and surrounds the capillary.

11. A mechanism as in claim 6 including means for vibrating the capillary.

12. A mechanism as in claim 1 in which the support tray includes cleaning wells for selectively receiving and cleaning the capillary.

13. A mechanism as in claim 6 in which the support tray includes cleaning wells for selectively receiving said mixing means and said capillary.

14. A cytometer system in which sample liquid is drawn from a sample container in an array of sample containers past an analyzing volume in a capillary by immersing the suspended end of the capillary in the sample liquid and drawing the liquid sample past the analyzing volume including:
a mechanism for supporting the individual sample containers in a first position and sequentially translating individual sample containers to a position opposite the end of the suspended capillary; and
lifting supporting means to immerse the end of the capillary in the sample liquid in the selected containers so that the liquid can be drawn through the capillary, and lowering the supporting means to repeat the translation and lifting.

15. A cytometer system as in claim 4 including a stirrer adapted to stir the liquid in a sample container.
